(19) United States
(12) Patent Application Publication
(10) Pub. No.: US 2020/0164185 A1
(43) Pub. Date: May 28, 2020

(54) TOOL AND METHOD FOR BALLOON REWRAPPING

(71) Applicant: Cardinal Health Switzerland 515 Gmbh, Baar (CH)

(72) Inventor: Quentin Walsh, Livermore, CA (US)

(73) Assignee: Cardinal Health Switzerland 515 Gmbh, Baar (CH)

(21) Appl. No.: 16/088,496

(22) PCT Filed: Mar. 29, 2016
(86) PCT No.: PCT/IB2016/000538

§ 371 Date: Sep. 26, 2018

(51) Int. Cl. A61M 25/10

(52) U.S. Cl. CPC A61M 25/1038; A61M 2207/10; A61M 2025/1081

(57) ABSTRACT

This disclosure is directed to a rewrapping tool (20) for returning a balloon of a dilatation catheter to a wrapped profile following inflation. The tool includes a body (22) extending along a longitudinal axis L-L defining a first lumen (24) and a channel (28) communicating along the length of the first lumen. The channel is to be slid over a catheter shaft of the dilatation catheter at a position proximal to the balloon so that the catheter shaft is coaxial with the first lumen and the catheter shaft may be pulled in a proximal direction so that the balloon is drawn through the first lumen to return the balloon to a wrapped profile.

### FIELD OF THE PRESENT DISCLOSURE

This disclosure generally relates to a catheter having an inflatable balloon for performing a dilatation procedure within a patient's vascular system and more particularly to a tool and method for returning a balloon that has been inflated to a wrapped profile.

### BACKGROUND

Catheter systems are used in a variety of therapeutic applications, including many vascular treatments due to the desire to provide minimally invasive procedures. Notably, a catheter may be provided with an inflatable balloon at the distal end for performing angioplasty, such as percutaneous transluminal coronary angioplasty (PTCA). Patency may be restored by inflating a balloon positioned in a blood vessel that is blocked or narrowed by a lesion or stenosis. It will be appreciated that a number of other procedures also employ a catheter having an inflatable balloon. The balloons used in such catheters are formed from relatively inelastic materials in order to positively control the outer diameter of the balloon once expanded. Accordingly, the balloon typically has wings or flaps of excess material when uninflated. During manufacture, the flaps are tightly wound around the catheter shaft to minimize the insertion profile to facilitate advancing the distal end of the catheter through a catheter sheath introducer (CSI) to gain access to the patient's vasculature.

In some situations, it would be desirable to utilize a dilatation catheter multiple times. Typically, the CSI is chosen to have the minimum diameter that will accommodate the dilatation catheter being employed. As such, there may be relatively little clearance between the inner diameter of the CSI and the balloon profile when wrapped. After the balloon has been inflated once, it may prove challenging to return the balloon to its wrapped profile. Correspondingly, re-inserting the dilatation catheter through the CSI may be considerably more difficult.

Conventional attempts to address this problem have involved the use of rewrapping tool that is shaped in the form of a tube having a lumen that is sized to compress the deflated balloon to a wrapped profile. The rewrapping tool may be provided separately from the dilatation catheter or it may be preloaded on a proximal portion of the shaft. By drawing the balloon through the lumen, the operator may rewrap the flaps around the catheter shaft, ideally returning the balloon to a profile that is close to its manufactured state. However, a number of deficiencies are associated with these techniques. If the rewrapping tool is provided as a separate device, the operator must thread the distal end of the catheter through the lumen and advance the balloon into the lumen. This operation involves a pushing force and the catheter shaft may not have sufficient column strength successfully pass the balloon through the lumen without buckling. Additionally, since the lumen of the rewrapping tool typically has a diameter close to that of the CSI, it is of comparable difficulty to simply re-introduce the dilatation catheter, obviating the motivation for employing the rewrapping tool.

Implementations in which the rewrapping tool is preloaded on the proximal catheter shaft insertion initially avoid these drawbacks, as the user rewraps the balloon by pulling the distal end of the catheter with the balloon through the lumen without the need to push the balloon through the lumen. Unfortunately, the benefit is only available once. A single pass of the balloon through the lumen of the rewrapping tool may not be sufficient to achieve the desired insertion profile. Further, it may be desirable to use the catheter more than twice. In either situation, the operator must now push the balloon through the lumen, which presents the difficulties discussed above.

Accordingly, the present inventor has recognized that there is a need in the art for a tool for rewrapping the balloon of a catheter mapping. Similarly, the inventor has recognized a need in the art for a tool that allows an operator to pull a deflated balloon through a lumen sized to return the balloon to a wrapped profile that may be employed multiple times without pushing the balloon through the lumen. Still further, the inventor has recognized a need in the art for a method of using such a tool that facilitates introduction of a balloon catheter through an introducer multiple times. The techniques of this disclosure as described in the following materials satisfy these and other needs.

### SUMMARY

The present disclosure is directed to a rewrapping tool for returning a balloon of a dilatation catheter to a wrapped profile following inflation having a body extending along a longitudinal axis, a first lumen defined by the body and having a length that extends between opposing ends of the body, and a channel communicating along the length of the first lumen, wherein the channel has a width and a depth matched to an inner diameter of the first lumen. The channel may be defined by opposing surfaces of a guide that projects from the body. The opposing surfaces of the guide may be formed from material thinner than a remainder of the body. The channel may have a width in the range of approximately 0.001″ to 0.005″ less than the inner diameter of the first lumen and the channel may have a depth greater than the inner diameter of the first lumen.

In one aspect, the rewrapping tool may have a second lumen defined by the body and having a length that extends between opposing ends of the body, wherein the channel communicates along the length of the second lumen and wherein the second lumen has an inner diameter greater than the inner diameter of the first lumen. The inner diameter of the second lumen may accommodate a deflated balloon of a dilatation catheter having a catheter shaft with an outer diameter that corresponds to the inner diameter of the first lumen.

In one aspect, the rewrapping tool may also have at least one intermediate lumen defined by the body and having a length that extends between opposing ends of the body, wherein the channel communicates along two lengths of the intermediate lumen and wherein the intermediate lumen has an inner diameter greater than the inner diameter of the first lumen and less than the inner diameter of the second lumen.

This disclosure also includes a kit for retuning a balloon of a dilatation catheter that has been inflated to a wrapped profile including a dilatation catheter having a catheter shaft extending along a longitudinal axis with an outer diameter and an inflatable balloon disposed around a distal portion of the catheter shaft and a rewrapping tool having a body extending along a longitudinal axis, a first lumen defined by the body and having a length that extends between opposing ends of the body, and a channel communicating along the length of the first lumen, wherein the channel has a width and a depth matched to an inner diameter of the first lumen and wherein the inner diameter of the first lumen accommodates the catheter shaft and compresses the balloon to a wrapped profile.

This disclosure also includes a method for rewrapping a balloon of a dilatation catheter involving providing a rewrapping tool having a body extending along a longitudinal axis, a first lumen defined by the body and having a length that extends between opposing ends of the body, and a channel communicating along the length of the first lumen, wherein the channel has a width and a depth matched to an inner diameter of the first lumen, sliding the channel over a catheter shaft of the dilatation catheter at a position proximal to the balloon so that the catheter shaft is coaxial with the first lumen and pulling the catheter shaft in a proximal direction so that the balloon is drawn through the first lumen to return the balloon to a wrapped profile. Sliding of the channel over the catheter shaft at a position proximal to the balloon and drawing the balloon through the first lumen may be repeated until a desired wrapped profile is obtained. As desired, the first lumen may be compressed as the balloon is drawn through. In addition, the rewrapping tool may be rotated slightly as the balloon is drawn through to assist in rewrapping the balloon to the desired wrapped profile.

In one aspect, the rewrapping tool has a second lumen defined by the body with an inner diameter greater than the inner diameter of the first lumen and a length that extends between opposing ends of the body and the channel communicates along the length of the second lumen and the method may include introducing the balloon through the second lumen and advancing the catheter shaft distally until the rewrapping tool is at the position proximal to the balloon. Sliding the channel over the catheter shaft may move the catheter shaft from being coaxial with the second lumen to being coaxial with the first lumen.

In one aspect, the rewrapping tool also has at least one intermediate lumen defined by the body with an inner diameter greater than the inner diameter of the first lumen and a length that extends between opposing ends of the body and the channel communicates along two lengths of the second lumen and the method may include sliding the channel over the catheter shaft of the dilatation catheter at a position proximal to the balloon so that the catheter shaft is coaxial with the intermediate lumen and pulling the catheter shaft in a proximal direction so that the balloon is drawn through the intermediate lumen prior to drawing the balloon through the first lumen.

### BRIEF DESCRIPTION OF THE DRAWINGS

Further features and advantages will become apparent from the following and more particular description of the preferred embodiments of the disclosure, as illustrated in the accompanying drawings, and in which like referenced characters generally refer to the same parts or elements throughout the views, and in which:

FIG. 1 is a top plan view of a dilatation catheter having an inflatable balloon suitable for use with the techniques of this disclosure.

FIG. 2 is an elevational view of the balloon of a dilatation catheter in a wrapped profile, according to one embodiment.

FIG. 3 is schematic cross section of the balloon in the wrapped profile of FIG. 2.

FIG. 4 is an elevational view of a single lumen rewrapping tool, according to one embodiment.

FIGS. 5-7 schematically illustrate operations that may be performed in rewrapping a balloon using the rewrapping tool of FIG. 4, according to one embodiment.

FIG. 8 is an elevational view of a dual lumen rewrapping tool, according to one embodiment.

FIGS. 9-12 schematically illustrate operations that may be performed in rewrapping a balloon using the rewrapping tool of FIG. 8, according to one embodiment.

FIG. 13 is an elevational view of a rewrapping tool having an intermediate lumen, according to one embodiment.

### DETAILED DESCRIPTION

At the outset, it is to be understood that this disclosure is not limited to particularly exemplified materials, architectures, routines, methods or structures as such may vary. Thus, although a number of such options, similar or equivalent to those described herein, can be used in the practice or embodiments of this disclosure, the preferred materials and methods are described herein.

It is also to be understood that the terminology used herein is for the purpose of describing particular embodiments of this disclosure only and is not intended to be limiting.

The detailed description set forth below in connection with the appended drawings is intended as a description of exemplary embodiments of the present disclosure and is not intended to represent the only exemplary embodiments in which the present disclosure can be practiced. The term “exemplary” used throughout this description means “serving as an example, instance, or illustration,” and should not necessarily be construed as preferred or advantageous over other exemplary embodiments. The detailed description includes specific details for the purpose of providing a thorough understanding of the exemplary embodiments of the specification. It will be apparent to those skilled in the art that the exemplary embodiments of the specification may be practiced without these specific details. In some instances, well known structures and devices are shown in block diagram form in order to avoid obscuring the novelty of the exemplary embodiments presented herein.

For purposes of convenience and clarity only, directional terms, such as top, bottom, left, right, up, down, over, above, below, beneath, rear, back, and front, may be used with respect to the accompanying drawings. These and similar directional terms should not be construed to limit the scope of the disclosure in any manner.

Unless defined otherwise, all technical and scientific terms used herein have the same meaning as commonly understood by one skilled in the art to which the disclosure pertains.

Finally, as used in this specification and the appended claims, the singular forms “a, “an” and “the” include plural referents unless the content clearly dictates otherwise.

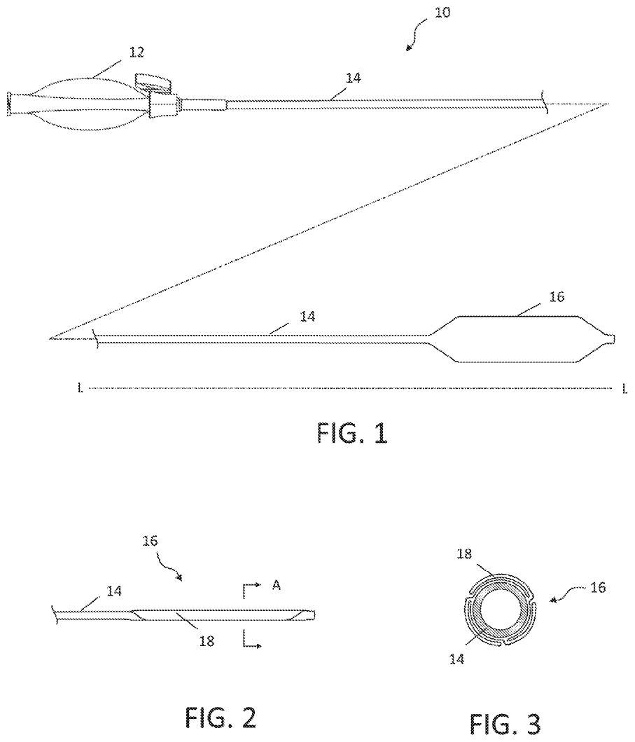

As noted above, this disclosure is directed to catheters having an inflatable balloon, such as dilatation catheters. More particularly, tools and methods are described for facilitating the rewrapping of the balloon multiple times. For context, an exemplary balloon dilatation catheter is illustrated in FIG. 1 by reference numeral 10. It includes a -hub assembly 12 of generally known structure for manipulating catheter 10 from a location outside of the patient's body in a manner which is generally known in the art. An elongated shaft 14 extends from hub assembly 12 and has an end assembly including an inflatable balloon 16. A corresponding longitudinal axis is indicated by L-L. In the context of this disclosure, proximal refers to portions of catheter 10 that are relatively closer to the operator, such as hub assembly 12, and distal refers to portions of catheter 10 that are relatively farther away from the operator, such as balloon 16. A port on hub assembly 12 communicates through a lumen extending through shaft 14 with one or more openings within balloon 16, so that inflation fluid may be supplied to expand balloon 16, which may be made of a material suitable for a dilatation balloon, such as a polymeric material, and in accordance with an appropriate molding approach for that material.

Balloon 16 is shown in FIG. 1 in a substantially inflated configuration. To control the maximum diameter achieved upon expansion, balloon 16 may be formed from a substantially inelastic or non-compliant material. Accordingly, when deflated as shown in the detail view of FIG. 2, flaps 18 may be formed from the balloon material that wrap around catheter shaft 14 to minimize the insertion profile of balloon 16. This configuration is schematically shown in FIG. 3, which is a cross section taken at A-A of FIG. 2. Any number of suitable techniques may be employed during manufacture to facilitate formation of the wrapped configuration of balloon 16, including forming fold lines in the balloon material and/or heat setting the wrapped configuration. However, after catheter 10 has been employed and balloon 16 inflated, it may be difficult to return balloon 16 to a wrapped configuration with an insertion profile equivalent to that achieved during manufacture. Correspondingly, the rewrapping tools and methods described in this disclosure may aid rewrapping a balloon that has been inflated.

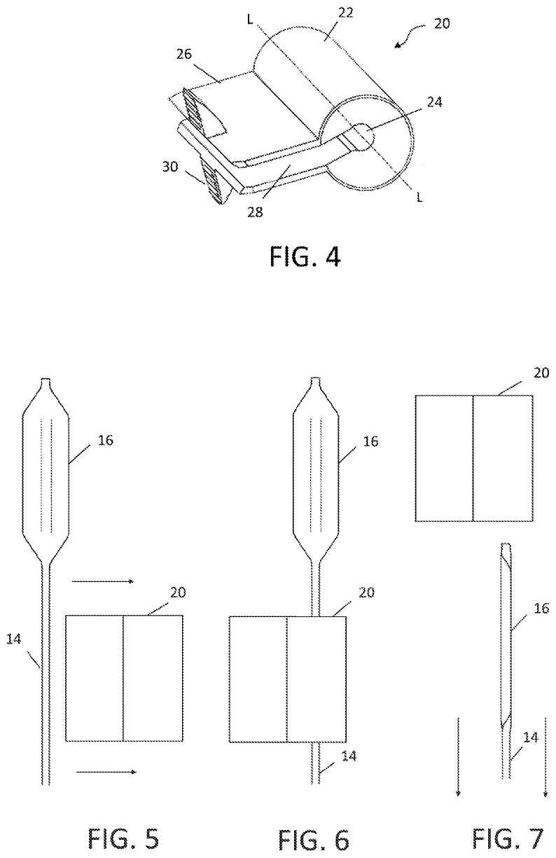

In one embodiment shown in FIG. 4, a rewrapping tool 20 according to this disclosure may include a body 22 that extends along a longitudinal axis L-L. The conventions regarding proximal and distal also apply to descriptions of rewrapping tool 20, such that proximal refers to portions of rewrapping tool 20 that are relatively closer to the operator and distal refers to portions of rewrapping tool 20 that are relatively farther away from the operator when it is being used to return balloon 16 to a wrapped profile. Body 22 defines a first lumen 24 having an inner diameter that approximates a desired insertion diameter for a given catheter design. In one aspect, this may be in the range of approximately 0.001″ to 0.005″ greater than the outer diameter of the catheter shaft. As desired, one or both ends of first lumen 24 may be concave or flared to facilitate passage of the balloon. A guide 26 that extends along the longitudinal axis L-L of body 22 may have opposing surfaces to define a channel 28 that also extends along the longitudinal axis L-L of body 22 and communicates with first lumen 24. Channel 28 allows rewrapping tool 20 to be placed on the catheter shaft so that the shaft is coaxial with first lumen 24 at a position proximal of the balloon. Grips 30 on opposing edges of guide 26 may be provided as desired to help position channel 28 over the catheter shaft. Guide 26 may project from body 22 a suitable amount to aid placing rewrapping tool 20 around the catheter shaft. For example, guide 26 may project from body 22 so that channel 28 correspondingly has a depth of at least a distance corresponding to the inner diameter of first lumen 24. Each portion of guide 26 may be formed from relatively thinner material so that they may deflect to accommodate passage of the catheter shaft as it is being position coaxial with first lumen 24.

The dimensions of channel 28 and first lumen 24 are matched to correspond to the characteristics of the given catheter with which rewrapping tool 20 will be used. For example, channel 28 may have a width equal to or less than the diameter of first lumen 24. In one aspect, channel 28 may have a width in the range of approximately 0.001″ to 0.005″ less than the diameter of lumen 24. As such, the width of channel 28 may be approximately the same or somewhat narrower than the outer diameter of the catheter shaft it is being used with, which is less than the diameter of balloon 16 in its wrapped profile. When the channel width is narrower, rewrapping tool 20 may be deflected slightly to accommodate introduction of the catheter shaft, creating a snap fit connection when the catheter shaft is fully inserted and coaxially aligned with first lumen 24.

Accordingly, rewrapping tool 20 may be formed of a suitable material, such a polymeric material, having a desired compliance with a relatively low durometer. Further, rewrapping material 20 may also be compressed as the balloon is being pulled through first lumen 24 to impart an enhanced rewrapping force. Exemplary materials include, without limitation, polyethylenes such as high density polyethylene (HDPE) and fluoropolymers such as polytetrafluoroethylene (PTFE). Suitable materials may be relatively lubricious to facilitate pulling the balloon through the first lumen. The length of body 22 may be long enough to allow ready manipulation by the operator and it may also be desirable to minimize the length of body 22 to reduce friction with the balloon as it is being drawn through, while maintaining sufficient length to be manipulated by the operator. As an example and without limitation, body 22 may have a length in the range of approximately 2 cm to 5 cm.

One suitable technique for employing rewrapping tool 20 is schematically illustrated in FIGS. 5-7. Beginning with FIG. 5, rewrapping tool 20 may be positioned adjacent catheter shaft 14 proximally of balloon 16. Catheter shaft 14 is slid through channel 28 until it is coaxially aligned with first lumen 24 as indicated by FIG. 6. From this position, catheter shaft 14 may be pulled proximally such that it translates in a substantially linear path along the longitudinal axis L-L with little or no buckling, drawing balloon 16 through first lumen 24, causing the balloon to return to a wrapped profile. In some instances, the operator may rotate catheter shaft 14 as balloon 16 is being drawn through first lumen 24 to aid the wrapping of the flaps. Alternatively or in addition, in some instances the operator may slightly rotate the wrapping tool 20 as balloon 16 is being drawn through first lumen 24 to aid the wrapping of the flaps. The sequence of operations represented by FIGS. 5-7 may be repeated as necessary until a desired insertion profile is achieved or between procedures in which balloon 16 is inflated.

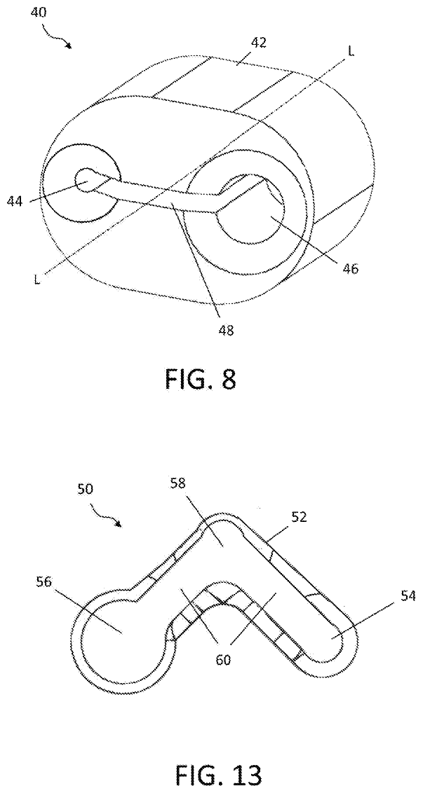

Another embodiment is schematically depicted in FIG. 8 as represented by rewrapping tool 40. This embodiment may employ similar construction and materials. As shown, rewrapping tool 40 may have a body 42 that extends along a longitudinal axis L-L defining first lumen 44 with an inner diameter that approximates a desired insertion diameter for a given catheter design. Body 42 also defines a second lumen 46 having an inner diameter larger than the inner diameter of first lumen 44. For example, in an embodiment with a catheter that is used with a CSI having a diameter of 0.0735″, the diameter of the first lumen 44 should be approximately 0.075″ and the diameter of the second lumen 46 should be 40-50% larger than the diameter of the first lumen 44, which in this instance, should be approximately 0.100″. One or both ends of first lumen 44 and/or second lumen 46 may be concaved or flared to facilitate passage of the balloon. Notably, second lumen 46 may have an inner diameter sized to accommodate a balloon in a deflated, but unwrapped configuration. Body 42 also defines a channel 48 that communicates between first lumen 44 and second lumen 46.

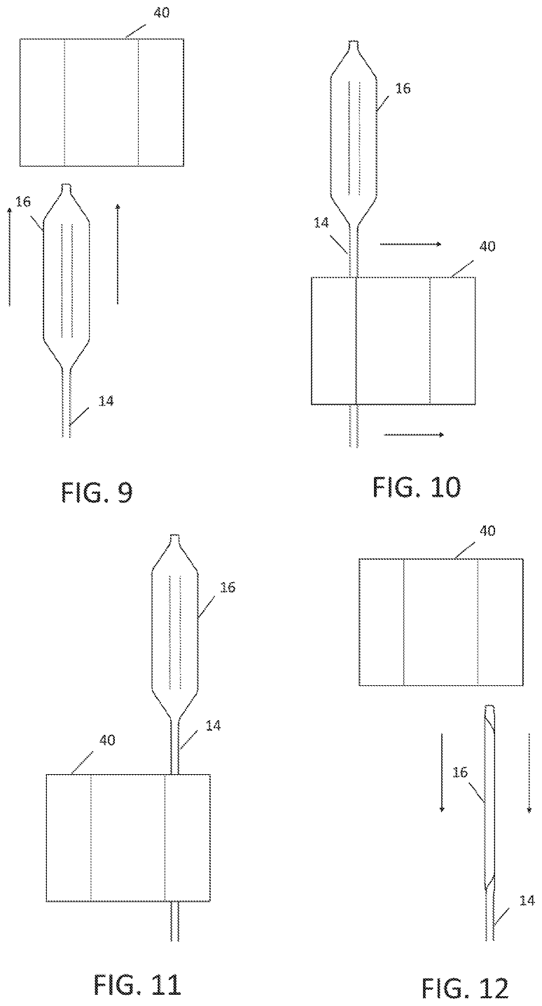

To illustrate the use of rewrapping tool 40, FIGS. 9-12 schematically depict operations that may be performed to rewrap a balloon after it has been inflated. Beginning with FIG. 9, balloon 16 may be pushed through second lumen 46 in a distal direction. As noted above, second lumen 46 may have an inner diameter that easily accommodates balloon 16 when deflated but not wrapped. Once catheter shaft 16 has been advanced through second lumen 46 until rewrapping tool 40 is positioned proximally of balloon 16 as shown in FIG. 10, catheter shaft 14 may be slid through channel 48 until it is coaxially aligned with first lumen 44 as indicated by FIG. 11. Now catheter shaft 14 may be pulled proximally such that it translates in a substantially linear path along the longitudinal axis L-L with little or no buckling, drawing balloon 16 through first lumen 44, causing the balloon to return to a wrapped profile. As above, the sequence of operations represented by FIGS. 9-12 may be repeated as necessary until a desired insertion profile is achieved or between procedures in which balloon 16 is inflated.

Yet another embodiment is schematically depicted in FIG. 13. As shown, rewrapping tool 50 may have a body 52 that defines first lumen 54 with an inner diameter that approximates a desired insertion diameter for a given catheter design. Body 52 defines a second lumen 56 having an inner diameter larger than the inner diameter of first lumen 54, such as an inner diameter sized to accommodate a balloon in a deflated, but unwrapped configuration. Body 52 also defines a third lumen 58 having an inner diameter intermediate between first lumen 54 and second lumen 56. A continuous channel 60 communicates between first lumen 44, third lumen 48 and second lumen 46.

Use of rewrapping tool 50 may be similar to that of rewrapping tool 40, in that balloon 16 may be pushed through second lumen 56 in a distal direction so that catheter shaft may be slid through channel 60 until it is coaxial with first lumen 54, allowing balloon 16 to be drawn through and rewrapped. However, one or more intermediate rewrapping operations may be performed as desired by positioning catheter shaft 14 so that it is coaxial with third lumen 58. Accordingly, balloon 16 may be drawn through third lumen 58 to begin the rewrapping process. Since the inner diameter of third lumen 58 is greater than the inner diameter of first lumen 54, it may be easier to first draw balloon through third lumen 58 one or more times to provide a partially wrapped configuration that has a reduced profile as compared to the deflated but unwrapped balloon. This intermediate stage may facilitate the subsequent drawing of balloon 16 through first lumen 54. In other embodiments, any suitable number of intermediate lumens may be provided so that the rewrapping tool has four or more lumens, all connected by a continuous channel. Further, the lumens may be offset as shown in FIG. 13 in a V-shaped configuration or the lumens may be positioned in line.

The preceding description has been presented with reference to presently disclosed embodiments of this disclosure. Workers skilled in the art and technology to which this disclosure pertains will appreciate that alterations and changes in the described structure may be practiced without meaningfully departing from the principal, spirit and scope of this disclosure. As understood by one of skilled in the art, the drawings are not necessarily to scale. Accordingly, the foregoing description should not be read as pertaining only to the precise structures described and illustrated in the accompanying drawings, but rather should be read consistent with and as support to the following claims which are to have their fullest and fair scope.

What is claimed is:

1. A rewrapping tool for returning a balloon of a dilatation catheter to a wrapped profile following inflation, comprising a body extending along a longitudinal axis, a first lumen defined by the body and having a length that extends between opposing ends of the body, and a channel communicating along the length of the first lumen, wherein the channel has a width and a depth matched to an inner diameter of the first lumen.

2. The rewrapping tool of claim 1, wherein the channel is defined by opposing surfaces of a guide that projects from the body.

3. The rewrapping tool of claim 2, wherein the opposing surfaces of the guide are formed from material thinner than a remainder of the body.

4. The rewrapping tool of claim 1, wherein the channel has a width in the range of approximately 0.001″ to 0.005″ less than the inner diameter of the first lumen.

5. The rewrapping tool of claim 1, wherein the channel has a depth greater than the inner diameter of the first lumen.

6. The rewrapping tool of claim 1, further comprising a second lumen defined by the body and having a length that extends between opposing ends of the body, wherein the channel communicates along the length of the second lumen and wherein the second lumen has an inner diameter greater than the inner diameter of the first lumen.

7. The rewrapping tool of claim 6, wherein the inner diameter of the second lumen accommodates a deflated balloon of a dilatation catheter having a catheter shaft with an outer diameter that corresponds to the inner diameter of the first lumen.

8. The rewrapping tool of claim 6, further comprising at least one intermediate lumen defined by the body and having a length that extends between opposing ends of the body, wherein the channel communicates along two lengths of the intermediate lumen and wherein the intermediate lumen has an inner diameter greater than the inner diameter of the first lumen and less than the inner diameter of the second lumen.

9. A kit for retuning a balloon of a dilatation catheter that has been inflated to a wrapped profile, comprising:
a dilatation catheter having a catheter shaft with an outer diameter and an inflatable balloon disposed around a distal portion of the catheter shaft; and
a rewrapping tool having a body extending along a longitudinal axis, a first lumen defined by the body and having a length that extends between opposing ends of the body, and a channel communicating along the length of the first lumen, wherein the channel has a width and a depth matched to an inner diameter of the first lumen and wherein the inner diameter of the first lumen accommodates the catheter shaft and compresses the balloon to a wrapped profile.

10. The kit of claim 9, wherein the channel is defined by opposing surfaces of a guide that projects from the body.

11. The kit of claim 9, wherein the channel has a width in the range of approximately 0.001″ to 0.005″ less than the inner diameter of the first lumen.

12. The kit of claim 9, wherein the channel has a depth greater than the inner diameter of the first lumen.

13. The kit of claim 9, wherein the rewrapping tool further comprises a second lumen defined by the body and having a length that extends between opposing ends of the body, wherein the channel communicates along the length of the second lumen and wherein the second lumen has an inner diameter greater than the inner diameter of the first lumen, such that the inner diameter of the second lumen accommodates the balloon when deflated but not wrapped.

14. The kit of claim 13, wherein the rewrapping tool further comprises at least one intermediate lumen defined by the body and having a length that extends between opposing ends of the body, wherein the channel communicates along two lengths of the intermediate lumen and wherein the intermediate lumen has an inner diameter greater than the inner diameter of the first lumen and less than the inner diameter of the second lumen.

15. A method for rewrapping a balloon of a dilatation catheter comprising:
providing a rewrapping tool having a body extending along a longitudinal axis, a first lumen defined by the body and having a length that extends between opposing ends of the body, and a channel communicating along the length of the first lumen, wherein the channel has a width and a depth matched to an inner diameter of the first lumen;
sliding the channel over a catheter shaft of the dilatation catheter at a position proximal to the balloon so that the catheter shaft is coaxial with the first lumen; and
pulling the catheter shaft in a proximal direction so that the balloon is drawn through the first lumen to return the balloon to a wrapped profile.

16. The method of 15, further comprising repeating the sliding of the channel over the catheter shaft at a position proximal to the balloon and drawing the balloon through the first lumen until a desired wrapped profile is obtained.

17. The method of claim 15, further comprising compressing the first lumen as the balloon is drawn through.

18. The method of claim 15, wherein the rewrapping tool has a second lumen defined by the body with an inner diameter greater than the inner diameter of the first lumen and a length that extends between opposing ends of the body and the channel communicates along the length of the second lumen, further comprising introducing the balloon through the second lumen and advancing the catheter shaft distally until the rewrapping tool is at the position proximal to the balloon.

19. The method of claim 18, wherein sliding the channel over the catheter shaft moves the catheter shaft from being coaxial with the second lumen to being coaxial with the first lumen.

20. The method of claim 18, wherein the rewrapping tool has at least one intermediate lumen defined by the body with an inner diameter greater than the inner diameter of the first lumen and a length that extends between opposing ends of the body and the channel communicates along two lengths of the second lumen, further comprising sliding the channel over the catheter shaft of the dilatation catheter at a position proximal to the balloon so that the catheter shaft is coaxial with the intermediate lumen and pulling the catheter shaft in a proximal direction so that the balloon is drawn through the intermediate lumen prior to drawing the balloon through the first lumen.
